(19) United States
(12) Patent Application Publication
(10) Pub. No.: US 2010/0173358 A1
(43) Pub. Date: Jul. 8, 2010

(54) METHOD FOR OBTAINING A VALUABLE PRODUCT, PARTICULARLY STARCH, FROM GRAIN FLOUR

(75) Inventors: Willi Witt, Tecklenburg (DE); Joachim Ringbeck, Oelde (DE); Conny Seemann, Oelde (DE); Dirk Lang, Ostenfelde (DE)

Correspondence Address: BARNES & THORNBURG LLP, 750-17TH STREET NW, SUITE 900, WASHINGTON, DC, 20006-4675, US

(73) Assignee: WESTFALIA SEPARATOR GMBH, Oelde (DE)

(21) Appl. No.: 12/525,919

(22) PCT Filed: Feb. 7, 2008
(86) PCT No.: PCT/EP2008/051500

§ 371 Date: Dec. 7, 2009

(30) Foreign Application Priority Data
Feb. 9, 2007 (DE) 10 2007 006 483.9

(51) Int. Cl. C07K 1/14; C13D 1/00; C12P 19/04; C12P 21/02; C12P 5/02; F02G 5/02

(52) U.S. Cl. 435/68.1; 127/43; 530/412; 435/101; 435/167; 60/685

(57) ABSTRACT

A process for obtaining a starch and a protein or both from grain flour, the process steps comprising: providing grain flour; mixing the grain flour with processed or fresh water to form a slurry; separating the slurry into it at least two fractions, the at least two fractions including two or more of a heavy A-starch fraction, a protein and B-starch fraction, and a pentosan fraction; and generating a biogas from at least one of the fractions from the separating step, the biogas being used for generating energy.

This application is a National Phase Application based upon and claiming the benefit of priority to PCT/EP2008/051500, filed on Feb. 7, 2008, which is based upon and claims the benefit of priority to German Patent Application No. DE 10 2007 006 483.9, filed on Feb. 9, 2007 the contents of both of which Applications are incorporated herein by reference.

### BACKGROUND OF THE INVENTION

### Background and Summary

The present disclosure relates to a method or process for obtaining a valuable product, such as starch and/or protein, from grain flour. The grain flour may be wheat flour.

For example, a process for obtaining starch from grain flour, such as wheat flour, according to the state of the art, is illustrated in FIG. 6.

Accordingly, the grain corn, from which the stalks and the chaff were removed, is supplied to a mill for further processing. For example, see Step 100 in FIG. 6.

In the mill, the grain is first slightly moistened, or conditioned, in order to break open the outer hull of the corn and expose the inner parts. The resulting bran, or hull, is separated from the still coarse flour and from the process by sifting. The bran can later be admixed to the created by-products, such as feed products, for example, coagulated protein and thin fibers, or can be partially split or directly burnt for obtaining energy.

Subsequently, the flour passes through several rolling steps until the necessary fineness of the flour has been reached, as required, by means of intermediate sifting in order to remove additional undesirable parts and ensure the required granulation and yield. Before the processing of the wheat flour to gluten and starch, as well as its by-products, the flour is conditioned by storage. Alternative measures for a conditioning are, for example, ventilation, fluidization or a direct enrichment with oxygen.

Following the conclusion of the grinding, the finished flour will be mixed with fresh water or process water at a ratio of 0.7 to 1.0 parts relative to 1 part flour for forming a wheat flour slurry which is free of dry flour particles. Subsequently, energy is mechanically fed to the slurry by way of a so-called high-pressure pump or a perforated-disk mixer in order to promote the matrix formation, for example, the cross-linking and agglomeration of the protein fractions for forming the actual wet gluten. Then, the slurry pretreated in this manner reaches a moderately stirred tank in which a dwell time of from 0 to 30 minutes is set. For example, see Step 101 in the Drawings.

In the next process step, the slurry is diluted again with a defined quantity of water, such as fresh or processed water, at a ratio of 1 part slurry to 0.5 to 1.5 parts water directly in front of the advantageously used 3-phase decanter in a so-called U-tube in the inverse current. In the 3-phase decanter, such as a horizontal centrifuge, the separation of the slurry will then take place mechanically into three different fractions under the influence of centrifugal forces. That is, the heavy A-starch fraction, or the underflow of the decanter, the protein phase and the B-starch phase, or the nozzle phase of the decanter, and the pentosan fraction, such as mucous substances or hemicelluloses. For example, see Step 102 in the Drawings, which is shown as a three-phase separation. The use of other separating processes, such as other centrifuges, is also conceivable according to the present disclosure.

Because of its special characteristics, such as visco-elasticity, the protein of wheat, also called “gluten”, represents a desired and valuable product which is easily sold in the foodstuff industry, such as, for example, to bakeries and meat or sausage products businesses, the feed product industry, for example, for fish farms and for many technical applications, such as glues and paper coating dyes.

For obtaining the valuable protein, the nozzle phase from the decanter is first subjected to a sifting at, for example, Steps 201 and 202 in order to separate the gluten from the B-starch. In this sifting step, the fine-grain starch, for example, the B-starch, and the fibers are separated from the gluten.

For example, starch with a fraction of less than 40% particles of a grain size of less than 10 μm is used here as the A-starch, and a granular starch, in whose fraction the portion of starch corns with a particle diameter of less than 10 μm is greater than 60%, is used as the B-starch. The B-starch product does not necessarily only consist of particles of the above type but may also contain additional constituents, such as a certain fraction of pentosans.

This sifting is predominantly carried out in 2 steps. In the subsequent process step, the gluten is subjected to a washing, for example, at Step 203, in order to remove additional enclosed “non-protein particles” as well as undesirable soluble constituents before it is then dehydrated, for example, at Step 204 and dried, for example, at Step 205.

The A-starch obtained from the 3-phase separation, like the protein, is further processed in an independent line.

A safety sifting first takes place at, for example, at Step 301, in order to remove and recover the smallest gluten particles.

Subsequently, a further sifting, at, for example, at Step 302, takes place during which the fiber parts are separated from the A-starch.

For the concentrating and washing, at, for example at Step 303, the A-starch is placed in a nozzle or disk separator, such as, for example, a vertical centrifuge.

Following the concentrating, washing of the A-starch, at, for example, Step 304, takes place by means of a 5- to 12-step hydrocyclone system or a 1- to 2-step or 3-phase separator line. This occurs before a further process step, for example, at Step 305, in which the starch is first dehydrated by means of a vacuum filter, a dehydration centrifuge or a decanter and is then dried, at, for example, Step 306.

The washed starch may also be subjected to a further treatment, such as a chemical and/or physical modification before the drying. Such further treatment is not illustrated.

In the course of the concentration in a 3-phase separator, at, for example, Step 303, the starch is split into two different fractions, such as a heavy coarse-grained starch fraction, called A-starch, and a finer starch fraction.

The fine-grain starch is carried away by way of the medium phase of the separator and, together with the sifted fine-grain starch from the protein sifting, is carried to an additional separator, at, for example, at Step 402. In this separator, the possibly sorted large-grain A-starch is recovered and fed back to the A-starch line, while the small-grain B-starch which, in turn, is discharged in the medium phase, is further processed in a “B-starch line”.

In this processing, the thus separated B-starch is obtained as a further by-product in that it is first dehydrated by means of a decanter, at, for example, Step 403, and is then dried, at, for example, Step 404.

The excess of process water, such as from Step 402 and possibly additional excess process water from other process steps are brought together, for example, at Step 501.

Then, liquid is separated from solids remaining in the process water by means of a phase separation, for example, at Step 502, which solids may then, for example, be dried and be used as feed products, at, for example, Step 504.

The dissolved and liquid constituents discharged with the top flow can be moved into an evaporating device, for example, at Step 503, in which the liquid flow is further concentrated before a further processing takes place, for example, by a biological waste water treatment. The remaining concentrate of the evaporating device is mixed with the bran from the grinding, and is mixed together with the concentrate from the 2-phase separation and is dried, for example, at Step 504.

Decanters, self-cleaning separators or 3-phase separators can be used in the phase separation process step 502.

Prior art relating to the general technological background, for example, a process for producing a high-protein and high-glucose starch hydrolyzate, is known from German Patent Document DE 41 25 968 A1. German Patent Document DE 196 43 961 A1 describes a use and a system for obtaining proteins from the flour of legumes. German Patent Document 100 21 229 A1 discloses a process for producing protein preparations.

The present disclosure relates to a further development of this known process such that the economic efficiency is increased.

The present disclosure thus relates to a process for obtaining a valuable product, such as a starch and/or protein, from grain flour. The steps of the process include: i.) grain flour being mixed with fresh or processed water for forming a slurry; ii.) the slurry is separated into at least two fractions, such as centrifugally into a heavy A-starch fraction, into a protein and B-starch fraction at a nozzle phase of the decanter, and into a pentosan fraction; iii.) biogas is generated from at least one of the fractions obtained during the separation of step ii., which biogas is used for generating energy; and iv.) the fraction used for generating the biogas is subjected to at least one liquefaction step, for example, at Step 505 and one phase separation, for example, at Step 506, and wherein the biogas is generated from the liquid phase of the phase separation.

According to illustrative embodiments of the present disclosure, the protein phase is further processed in the protein processing steps for forming a protein product, the A-starch fraction is further processed for forming an A-starch product and biogas is generated from the B-starch.

In addition, it is expedient for the B-starch with bran and the pentosan fraction from the three-phase separation, at, for example, at Step 102, to be processed for forming biogas.

Advantageously, the liquefaction and a phase separation are included in a process of a biogas system, and energy is obtained directly from poly- and oligosaccharides naturally occurring during the starch production.

The preceding heat treatment and enzymatic treatment, as well as the subsequent separation of the substances, such as proteins, phospholipoproteins, celluloses, which are very difficult to utilize microbiologically, represent a difference with respect to a “conventional” biogas system.

Overall, it is achieved that a short amount of time is required until the generating of biogas is concluded. As a result of the “splitting” into low-molecular sugars, the latter are easily made accessible to the acid-forming and ethanoic-acid-forming bacteria, for example, these can rapidly metabolize the offered substrate.

As a result, the required dwell times are low relative to the load in the reactors, and therefore the construction of the latter can be relatively small. A good high value is achieved regarding COD freight. In this manner, an economically and technically controllable and meaningful processing of one or more phases or fractions from the starch production process into biogas easily becomes possible.

A special advantage is the resulting use of byproducts from obtaining protein and starch for directly generating energy. So far, all products had either been sold directly or had been converted to other products, such as, for example, modification, saccharification, ethanol production. The obtained energy can, in turn, be returned directly into the system. On the one hand, as electric energy and/or, on the other hand, as thermal energy, such as, for engine-based cogeneration system, gas engine, gas turbine.

The water draining off the methane stage can advantageously be processed in a membrane system that follows. As such, the membranes stressed to a slight degree and high flow rates are obtained. The permeate obtained from the membrane system can be returned as process water into the system.

Concerning the background of biogas systems, reference is made to Konstandt, H. G. (1976) “Engineering, Operation and Economics of Methane Gas Fermentation”, Göttingen: Microbiol. Energy Conservation Seminar, and to Kleemann, M. & Meliβ, M. (1993), “Regenerative Energy Sources”, Second, completely revised edition, Berlin, Springer, which should also be used as an example with respect to numerical data of the specification. Reference is also made to German Patent Document DE 103 27 954 A1 which describes a process for producing ethanol from a biomass. German Patent Document DE 198 29 673 A1 suggests the treatment of waste water from oil seed and grain processing of rape, sunflower or olive oils, the separating of the solids and the utilizing of these solids for obtaining biogas.

Other aspects of the present disclosure will become apparent from the following descriptions when considered in conjunction with the accompanying drawings.

### BRIEF DESCRIPTION OF THE DRAWINGS

FIGS. 1 to 5 are diagrams of different embodiments of a process according to the present disclosure.

FIG. 6 is a diagram of a process according to the state of the art.

### DETAILED DESCRIPTION

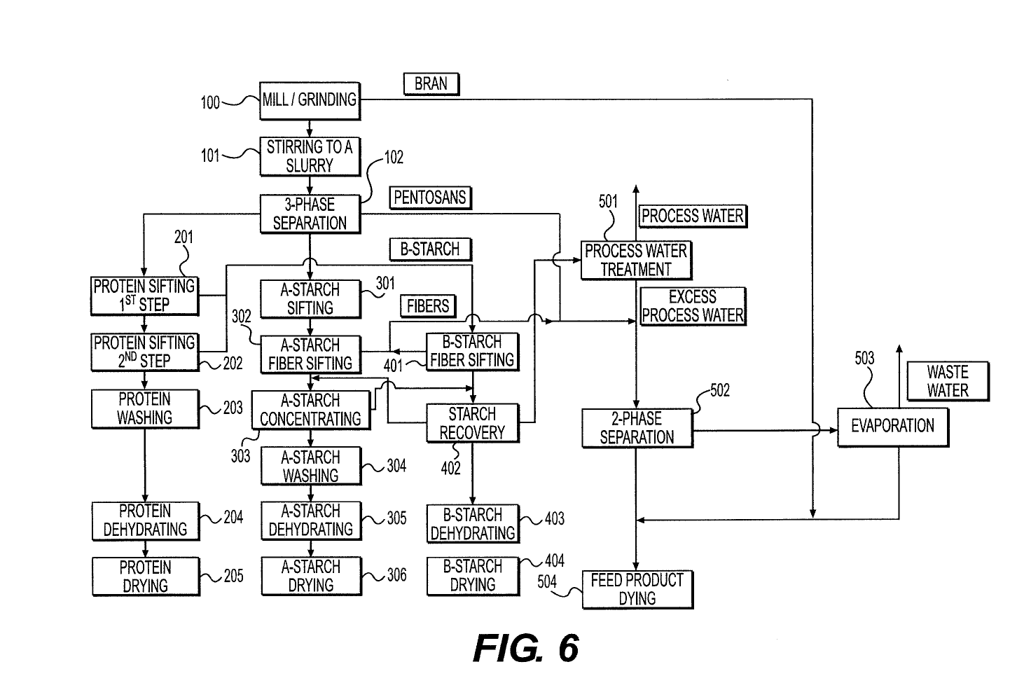

Analogous to FIG. 6, the processing of the grain and of the resulting flour respectively in Steps 100 to 102, 201 to 205 and 301 to 306 can take place in the manner shown in FIG. 6 or in the above-described process steps.

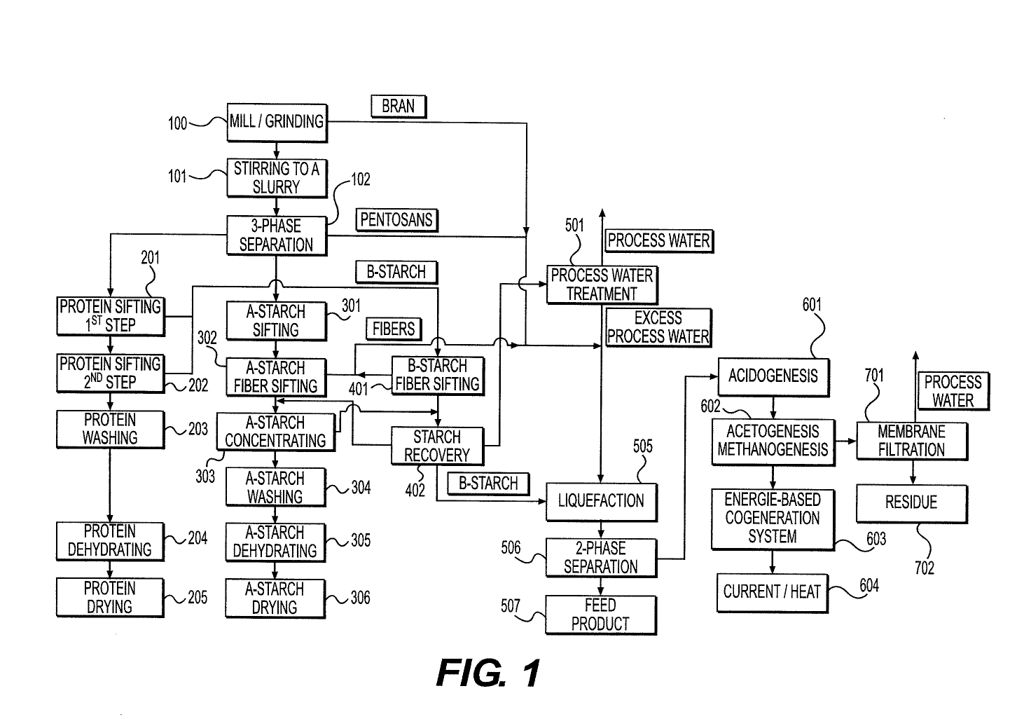

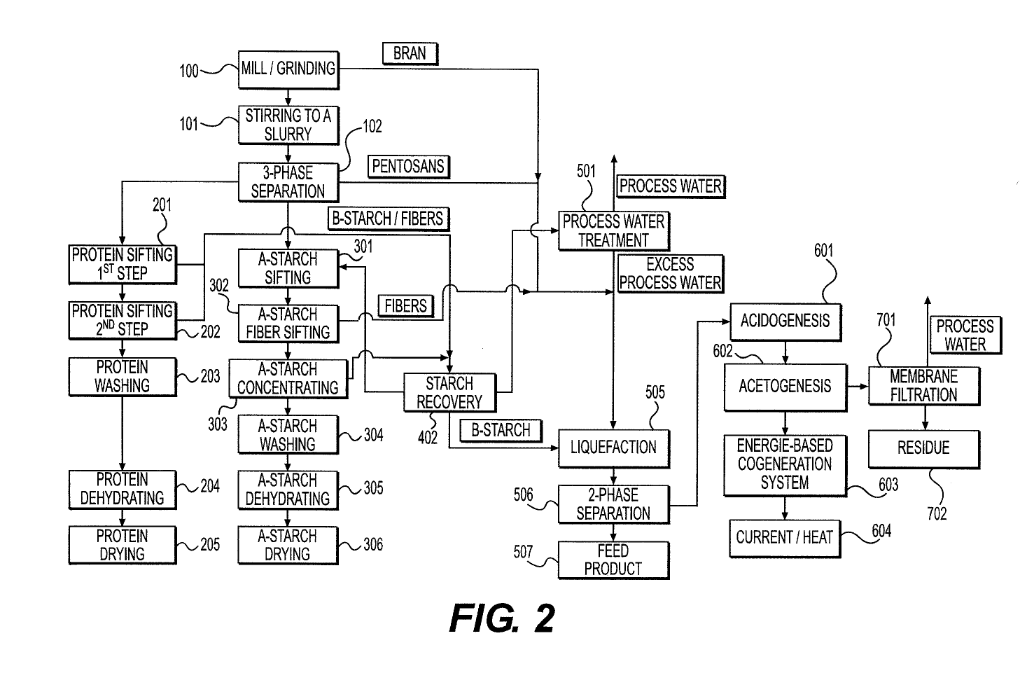

However, in contrast to FIG. 6, according to illustrative embodiments of the process of FIGS. 2 to 5, when the process is carried out, the B-starch is not obtained directly as a product but brought together with the substance flows from the 3-phase separation of: Step 102, the pentosans; the fiber sifting of Step 302 and possibly Step 401 as shown in, for example, FIGS. 1-5; the excess process water of Step 501; the bran from the grinding of Step 100; and, as a mixture, is subjected to a liquefaction at Step 505.

As illustrated, as an example, in FIG. 1, different substance flows from the process are brought together in the liquefaction at Step 505.

These are the pentosan fraction from Step 102 and the excess of process water, such as from Step 402 and starch recovery, as well as possibly additional process water excess from other process steps.

In the liquefaction at Step 505, the substances contained in the flows fed into the liquefaction are subjected to an enzymatic as well as to a thermal treatment in order to split the remaining macromolecular carbon compounds, such as starch, celluloses, and hemicelluloses, into smaller units and to coagulate and precipitate the remaining protein.

For the splitting of the macromolecular carbohydrates and the subsequent saccharification, various enzymes, such as cellulases, for example, Genencor 220 and SPEZYME FRED, for example, Genencor, are added which become effective at different temperature ranges. The temperature ranges may be, for example, I: 40° C.-60° C., or 45° C.-55° C., or 50° C., and II: 80° C.-95° C., or 85° C.-95° C., or, 90° C. During this step-by-step temperature treatment, the proteins are denatured in a parallel manner and precipitate together with the fine fibers and phospholipoproteins as a so-called protein coagulate.

Together with this coagulate, phosphorus, sulfur and nitrogen compounds are also precipitated, which microbiologically can be reduced only with difficulty and over an extended period of time. The separation of these substances is advantageous for a good efficiency of the biogas system, as well as for the splitting of poly- and oligosaccharides into low-molecular compounds.

Another advantage, according to the process of present disclosure, is the possibility of a good processing of the remaining waste water from the methane reactor to process water in a membrane filtration system because the danger of clogging the membranes is rather low.

In the subsequent process step of the phase separation, for example, at Step 506 using, for example, a decanter, self-cleaning separator or 3-phase separator, the thus precipitated solid constituents will then be separated from the liquid phase.

In such a case, the solids are the residual solid constituents which could not be influenced by the enzymes and heat, as well as the coagulated proteins and phospholipoproteins, such as protein coagulate.

This dehydrated mass can be further utilized as a feed product, a fertilizer or a combustion material, as suggested at Step 507.

Simultaneously, the content of P-, N- and S-compounds is thereby considerably reduced in the saccharified solution, which, advantageously, significantly improves a later anaerobic treatment.

The dissolved low-molecular sugars from the mechanical separation are moved into an acidification reactor in which they are microbiologically metabolized to different carbon acids and alcohols. The implementation of this process takes place, for example, by fermentative microorganisms of the pseudomonas, clostridium, lactobacillus and bacteroides species. In an illustrative embodiment according to the present disclosure, the dwell time in such a process step, for example, at Step 601, may be assumed to be approximately 2 days.

The metabolic products from the acidification step occurring in the acidogenesis are subsequently, in a second reactor, the so-called methane reactor, also microbiologically transformed to ethanoic acid, the syntrophomonas wolfei microorganism, for example, participating in Step 602, representing, methanogenesis.

The obtained ethanoic acid will then be anaerobically metabolized by methane-forming agents, such as methanobacterium bryantii, to methane and carbon dioxide. The duration of this process step or the dwell time amounts to approximately 10 days, the reactor having to handle a COD load of approximately 15-25 kg³.

The thus obtained gas mixture, or biogas, is collected and, in an engine-based cogeneration system, at, for example, Step 603, engine-based cogeneration system BHKW, and Step 604 energy generation converted to energy, such as to thermal and electric energy, for example, by means of a gas turbine or a gas engine.

During the anaerobic fermentation of the substrates in the methane reactor, a few residual substances and a little liquid still remain which have to be removed again from the reactor. In order to make the remaining water from the fermentation usable again, it is processed in a membrane system, for example, at Step 701. This system may be composed of one or more, for example, two or three steps.

It could therefore be possible, according to the present disclosure, to use only a single membrane step, or reverse osmosis.

When two membrane steps are used, for example, particles which have a diameter of >1 μm can be separated first in a first step, or micro-/ultrafiltration. The thus obtained permeate will then be largely demineralized in the 2^{nd} step by reverse osmosis, so that it can be used again as process water.

When three membrane steps are used, for example, particles which have a diameter of >1 μm can be separated first in a first step, or micro-/ultrafiltration. In view of the permeate of the first step, a low-pressure reverse osmosis step would be conceivable, according to the present disclosure, with the advantage of a rather low energy consumption, and a high-pressure reverse osmosis would be conceivable, according to the present disclosure, as a third step.

Because of the enriched mineral and nutrient contents, the remaining residues at, for example, Step 702 from the purification steps may possibly be sold as fertilizer.

The permeate can again be used as process water and can be returned, for example, into the process water treatment or collection system.

FIGS. 2 to 5 show different illustrative embodiments, according to the present disclosure, for carrying out the process for obtaining the energy carriers, the byproduct utilization, such as feed products, modified starch, as well as an added obtaining of process water.

FIG. 2 illustrates an implementation of the process in which the system part of Step 401 for the B-starch fiber sifting is removed from the process because the fibers are returned again to this product flow in the later process. This approach has the result that the recovered starch from the recovery separator, at Step 402, has to be conducted back in front of the fiber sifting of Step 302 of the A-starch so that the A-starch can be separated again from the fibers.

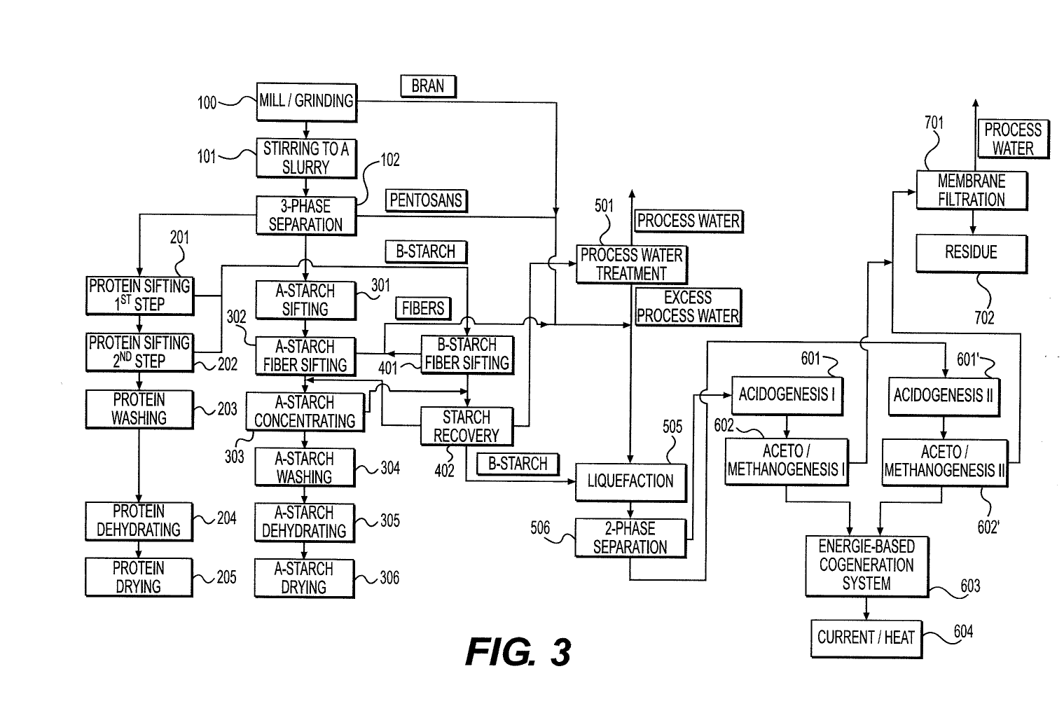

FIG. 3 describes an alternative use of the feed product obtained from variant B at Step 507. Instead of using these residual constituents as feed products, the possibility exists, according to the present disclosure, of fermenting these substances, such as proteins, or residual fibers, etc., also in a separate biogas system in the “Acidogenesis” at Step 601′ and Acetogenesis at Step 602′ which steps may be parallel to Steps 601 and 602, to obtain methane in order to increase the energy efficiency.

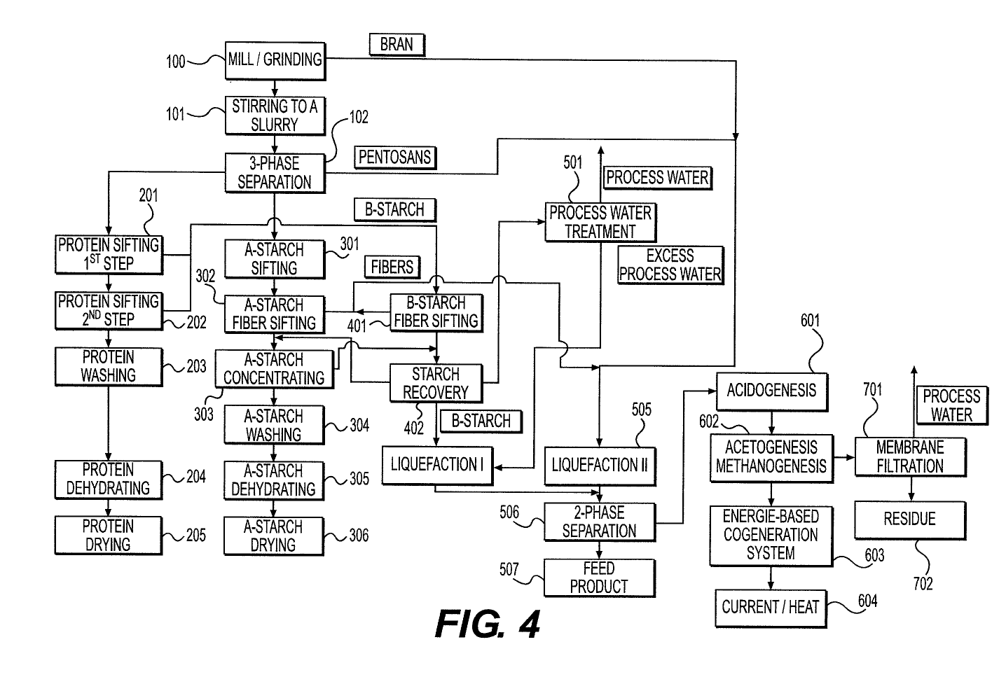

FIG. 4 illustrates another illustrative embodiment according to the present disclosure. In order to increase the effectiveness as a result of the specificity of the enzymes, the pentosans and the bran are moved into a separate liquefaction, at, for example, Step 505′, where special pentanases and cellulases are used.

The fine-grain starch and fine fibers from the recovery separator, the fiber sifting and the process water treatment are also moved into their own liquefaction, such as at Step 505.

The flows from the separated liquefaction Steps 505 and 505′ are brought together again before the mechanical separation of Step 506.

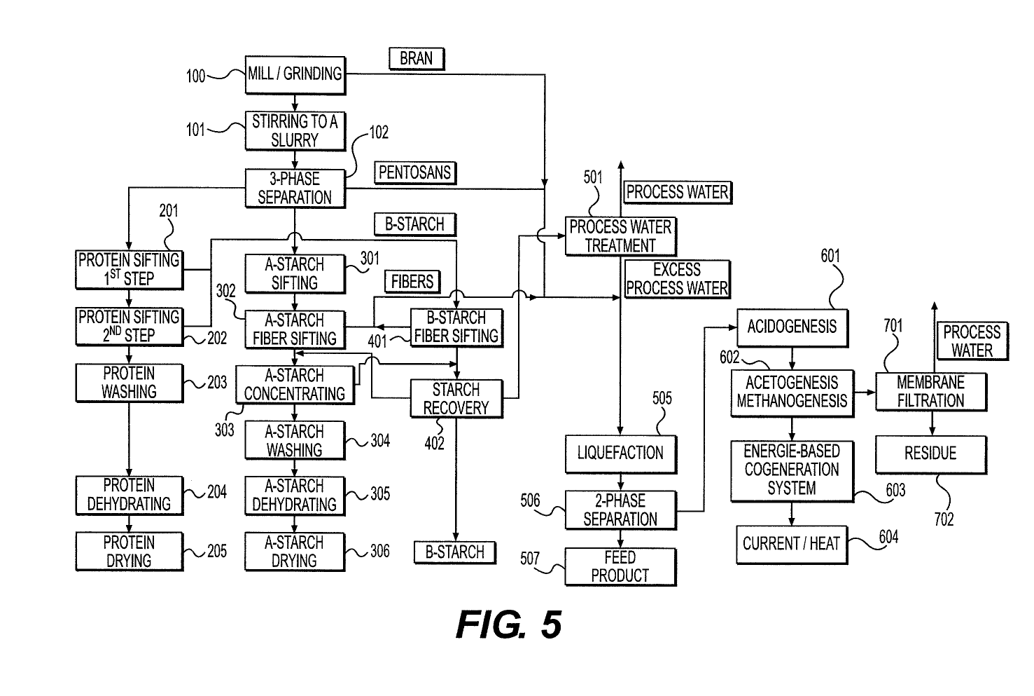

Furthermore, the process variant of FIG. 5 should be indicated as an additional alternative. When implementing the process of this illustrative embodiment, a portion of the energy generation is not carried out for the benefit of a further product.

In contrast to the preceding illustrative embodiments, the B-starch occurring in the course of the process is not used as an energy carrier in the gas fermentation but as a valuable product such as modified starch.

In the following, the energy balance of the illustrative process or processes, according to the present disclosure are considered as an example.

The following reaction equation is used as a starting or simplified basis for the theoretical analysis of the gas yield and the energy that can be obtained therefrom:

| | |
|---|---|
| 2 C₆H₁₂O₆→ 6 CH₄ + 6 CO₂ | Molar glucose |
| | mass 180 g/mol |
| | correspondingly 360 g/mol |
| | for saccharose |
| Molar methane mass 16 g/mol | |
| Spec. methane enthalpy 802 KJ/mol | |

Approximately 0.2667 kg methane is therefore obtained from 1 kilogram starch. This amount of methane has an energy value of 13.4 MJ. An energy quantity of 13.4 GJ can therefore be obtained per one ton of starch.

A medium-sized wheat starch facility processes approximately 10 tons of flour per hour, which corresponds to a grain quantity of approximately 12.5 t/h. For obtaining energy, approximately 2,900 kg usable carbohydrates are obtained from the above. A facility of this processing capacity can therefore theoretically produce approximately 10.8 MWh of energy in one hour.

The estimated energy demand of such a facility, without B-starch drying, fiber drying and evaporating system, amounts to approximately 307.5 KWh/t of flour electrically and 2.2 GJ/t of flour thermally, that is, steam.

When a realistic efficiency of η=0.3 is assumed for converting methane gas to electric energy, 326 KWh of electric energy per ton of flour can be obtained from the gas obtained from the starch.

Furthermore, when it is assumed that, by means of a coupling of power and heat, the lost energy during the generating of current can be converted to heat and finally steam, 2.74 GJ/t of flour as energy are still available for producing steam. With an efficiency of η=0.88, an energy quantity of 2.4 GJ is therefore obtained, which can influence the generating of steam.

It is illustrated that the required energy for the operation of the facility is covered from the obtained energy of the biogas production, and the latter could therefore be operated self-sufficiently with respect to energy.

For the purpose of comparison, the following values for the gas yield from biogas facilities can be found in literature:

| | |
|---|---|
| From carbohydrates | 790 Ln biogas/kg TS with a |
| | methane fraction of 50% |
| Energy content biogas | approximately 5 KWh/Nm³ |
| | (natural gas: approx. |
| | 10 KWh/Nm³) |

From 290 kg carbohydrates/t of flour, an energy quantity of approximately 1,145.5 KWh/t of flour can therefore be obtained, at facility capacity of 10 t/h corresponding to 11.45 MWh.

Ln: Standard liter

Nm³: Standard cubic meter

TS: Dry substance

Although the present disclosure has been described and illustrated in detail, it is to be clearly understood that this is done by way of illustration and example only and is not to be taken by way of limitation. The scope of the present disclosure is to be limited only by the terms of the appended claims.

1. A process for obtaining a starch and a protein or both from grain flour, the process steps comprising:
providing grain flour;
mixing the grain flour with processed or fresh water to form a slurry;
separating the slurry into at least two fractions, the at least two fractions including two or more of a heavy A-starch fraction, a protein and B-starch fraction, and a pentosan fraction;
generating a biogas from at least one of the at least two fractions from the separating step, the biogas being used for generating energy; and
subjecting the at least one fraction used for generating the biogas to at least one liquefaction step and one phase separation step such that the biogas is generated from a liquid phase of the phase separation step.

2-24. (canceled)

25. The process according to claim 1, wherein the protein and B-starch fraction is further processed to form a protein product, the A-starch fraction is further processed to form an A-starch product, and the biogas is generated from at least one or both of the B-starch fraction and the pentosan fraction.

26. The process according to claim 25, wherein the B-starch fraction together with a bran and the pentosan fraction are processed to form the biogas.

27. The process according to claim 1, wherein different substance flows from the process are brought together and subjected to a process water treatment step and a liquefaction step.

28. The process according to claim 27, wherein the pentosan fraction, an excess of processed water from a starch recovery step and additional processed water excess from other process steps are brought together in the process water treatment step.

29. The process according to claim 1, wherein the at least one fraction from which the biogas is generated is subjected to an enzymatic treatment in the liquefaction step in order to coagulate proteins and to split macromolecular carbon compounds into smaller units.

30. The process according to claim 29, wherein for the splitting of macromolecular carbon compounds and subsequent saccharification, enzymes are added to flows in the liquefaction step, which become effective at different temperature ranges.

31. The process according to claim 30, wherein the added enzymes to the flows in the liquefaction step, become effective at different temperature ranges including a first range from 40° C.-60° C. and a second range from 80° C.-95° C., so that, during a step-by-step temperature treatment, proteins are denatured in a parallel manner which are precipitated together with fine fibers and phospholipoproteins as a protein coagulate, and that, together with this coagulate, phosphorus, sulfur and nitrogen compounds are also precipitated.

32. The process according to claim 1, wherein, in the phase separation step, which follows the liquefaction step, solid constituents precipitated in the liquefaction step are separated from the liquid phase.

33. The process according to claim 32, wherein a dehydrated mass from the phase separation step is utilized as one of a feed product, fertilization or combustion material step.

34. The process according to claim 33, wherein the phase separation step takes place in one of a decanter, a self-cleaning separator, a 3-phase separator or by filtration.

35. The process according to claim 1, wherein dissolved substances from the phase separation step are subjected to an acidogenesis step.

36. The process according to claim 35, wherein the dissolved substances during the acidogenesis step, are brought into an acidification reactor, in which they are microbiologically metabolized to different carbon acids and alcohols.

37. The process according to claim 35, wherein a dwell time in the acidogenesis step amounts to fewer than 4 days.

38. The process according to claim 36, wherein the metabolized products from the acidogenesis step are subsequently, in a methane reactor, microbiologically transformed to ethanoic acid, and the obtained ethanoic acid is anaerobically metabolized by methane-forming agents to methane and carbon dioxide.

39. The process according to claim 38, wherein a duration of the acidogenesis step amounts to fewer than 14 days.

40. The process according to claim 39, wherein the methane reactor handles a COD load of approximately 15-25 kg/m³.

41. The process according to claim 1, wherein the obtained biogas is collected and, in an engine-based cogeneration system step and an energy generation step, is converted to energy by a gas engine.

42. The process according to claim 38, wherein liquid from the methane reactor is subjected to a filtration in an at least a one-step membrane filtration step.

43. The process according to claim 42, wherein particles with a larger diameter are first separated and second, an obtained permeate is demineralized by reverse osmosis such that the permeate can be used again in a processed water step.

44. The process according to claim 42, wherein particles with a larger diameter are first separated, and second, an obtained permeate is subjected to a low-pressure reverse osmosis, and third, is subjected to a high-pressure reverse osmosis.

45. The process according to claim 43, wherein the permeate is returned into the process water treatment step.

46. The process according to claim 26, wherein the pentosan fraction and the bran are processed in a first liquefaction step, and fine-grain starch and fine fibers are processed in a second liquefaction step in separate flows during the process.

47. The process according to claim 46, wherein the separate flows from the first and second liquefaction steps are brought together before the phase separation step.

48. The process according to claim 1, wherein the protein and B-starch fraction exit at a nozzle phase of a decanter used for this process.

49. The process according to claim 1, wherein the grain flour is wheat flour.

50. The process according to claim 29, wherein the enzymatic treatment is thermal treatment.

51. The process according to claim 29, wherein the macromolecular carbon compounds include starch.

52. The process according to claim 29, wherein the macromolecular carbon compounds include celluloses.

53. The process according to claim 29, wherein the macromolecular carbon compounds include hemicelluloses.

54. The process according to claim 30, wherein the enzymes include celluloses.

55. The process according to claim 30, wherein the enzymes include SPEZYME FRED.

56. The process according to claim 31, wherein the first temperature range is 45°-55° C.

57. The process according to claim 31, wherein the first temperature range is 50° C.

58. The process according to claim 31, wherein the second temperature range is 85°-95° C.

59. The process according to claim 31, wherein the second temperature range is 90° C.

60. The process according to claim 35, wherein the dissolved substances include low-molecular sugars.

61. The process according to claim 36, wherein the dissolved substances include low-molecular sugars.

62. The process according to claim 37, wherein the dwell time amounts to fewer than 2 days.

63. The process according to claim 38, wherein the methane-forming agents include methanobacterium bryantii.

64. The process according to claim 39, wherein the duration amounts to fewer than 10 days.

65. The process according to claim 41, wherein the gas engine is a gas turbine.
